(19) United States
(12) Patent Application Publication
(10) Pub. No.: US 2025/0249181 A1
(43) Pub. Date: Aug. 7, 2025

(54) IMPROVED NEEDLE SAFETY ASSEMBLY

(71) Applicant: TIP-TOP.COM LTD, Colchester (GB)

(72) Inventor: Barry P. Liversidge, Colchester (GB)

(73) Assignee: TIP-TOP.COM LTD, Colchester (GB)

(21) Appl. No.: 18/568,002

(22) PCT Filed: Jun. 10, 2022
(86) PCT No.: PCT/GB2022/051468

§ 371 Date: Dec. 7, 2023

(30) Foreign Application Priority Data
Jun. 11, 2021 (GB) 2108399.3
Feb. 14, 2022 (GB) 2201934.3

(51) Int. Cl. A61M 5/32

(52) U.S. Cl. CPC A61M 5/3245; A61M 2005/3249; A61M 2005/3253

(57) ABSTRACT

The safety needle assembly comprises a needle/cannula (112) mounted in a tubular housing (120). The needle has a patient end (114) and a non-patient end and an aim of the invention is to provide passive needle stick protection at the non-patient end. A further aim is to increase the reliability and to reduce fatigue on the spring means prior to use. The cannula (112) is located within the needle mount (160) which is located within the tubular housing (120). The shielding sleeve (130) provides the blocking means which retains the alignment of the cannula (110) along the longitudinal axis of the tubular member (120). In particular, the blocking means is arranged to counteract the pivoting force of the spring or an ability of the cannula to freely rotate in order to retain the cannula (112) in an operative position. In the present invention, the spring (170) and the needle mount (160) are arranged at a set position and the use of the safety needle assembly (110) with the medical injector (111) causes a shift from the set position. This shift enables the spring (170) to then rotate the needle (112) to the shielding position when the safety needle assembly (110) is detached from the medical injector (111).

### FIELD OF THE INVENTION

The present invention relates to a safety needle assembly for use with a medical injector, a drug delivery device comprising a safety needle assembly and a medical injector and a method of shielding a non-patient end of a needle in a safety needle assembly.

### BACKGROUND TO THE INVENTION

Needle stick injuries from drug delivery devices/safety engineered devices may be prevented by active systems and/or passive systems which provide two different types of safety feature activation methods. In active systems, a user is required to activate the safety mechanism and these may require one handed or two handed activation by the healthcare professional after use. However, in passive systems, no intervention is required from the user and the safety system is activated automatically and specifically does not need any action/steps from the user. In particular, in passive systems the safety features are initiated and activated automatically during use of the device.

A needle safety assembly may include a spring in order to urge the needle to move from an operative position to a shielding position. This spring is assembled directly into a loaded position and a blocking device is arranged to prevent the needle moving from the operative position to the shielding position until the needle has been used and the injection preformed. Accordingly, the spring may be maintained in the loaded position for a prolonged period. Such springs may be formed from a resilient material which is deformed from an unbiased position to provide the required urging force for the needle and the spring is thereby preloaded. Accordingly, the material must have properties which ensure that the spring capacity does not reduce significantly before the assembly has been used. In addition, the material of the spring must have such inherent properties that the spring does not fail in any way whilst being maintained in a deflected position for a prolonged period. Such properties may dictate the period within which the assembly must be used or may limit the urging force which can be initially preloaded.

In metal springs, the material may be able to maintain the preloaded force. However, metal springs cause problems during assembly and this is a significant problem for devices which are being assembled in high speed assembly processes. For example, coiled metal springs can nest and cause other issues during assembly. Springs formed from a plastics material provide advantages since they can be reliably and accurately manufactured and these can also be used in high speed assembly processes. However, any preloading of such resilient material may gradually reduce with time.

It is an aim of the present invention to overcome at least one problem associated with the prior art, whether referred to herein or otherwise.

### SUMMARY OF THE INVENTION

According to a first aspect of the present invention there is provided a safety needle assembly for use with a medical injector comprising;
a tubular housing removably attachable to the medical injector, the tubular housing extending in a longitudinal direction from a proximal end to a distal end, the proximal end being arranged for attachment to the medical injector;
a needle mount located within the tubular housing for directly or indirectly supporting a double ended needle having a patient end and a non-patient end, the needle mount being arranged to allow movement of the needle from an operative position to a shielding position;
a spring means to urge movement of the needle to the shielding position;
a releasable blocking means arranged to prevent movement of the needle from the operative position whereat the needle extends in a direction along a longitudinal axis of the tubular housing and whereby release of the blocking means allows movement of the needle to the shielding position;
the needle mount is a unitary component inserted into the tubular housing and positioned so as to rotate around an axis and the spring means is arranged to urge rotation of the needle about said axis; and
the blocking means engages the needle mount to maintain the needle in the operative position before use, thereafter attachment of the safety needle assembly to the medical injector and the use thereof causes the blocking means to disengage from the needle mount and allow the spring means to rotate the needle to the shielding position and to move the non-patient end of the needle towards the distal end of the tubular housing whereat the needle extends at an angle oblique to the longitudinal axis of the tubular housing to place the non-patient end of the needle at a location adjacent to an interior wall of the tubular housing;
characterised in that;
the spring means is integral to a mounting member, and
in the operative position before use, the mounting member and the needle mount are arranged at a set position relative to each other, thereafter use of the safety needle assembly with the medical injector causes a shift from the set position; said shift moves one of the mounting member or the needle mount relative to the other to enable the spring means to rotate the needle to the shielding position when the safety needle assembly is detached from the medical injector.

The mounting member may be arranged to move proximally relative to the needle mount in order to cause the shift from the set position to preload the spring means to generate a force sufficient to enable the spring means to rotate the needle to the shielding position when the safety needle assembly is detached from the medical injector. The mounting member may be arranged to move distally relative to the needle mount in order to cause the shift from the set position to preload the spring means to generate a force sufficient to enable the spring means to rotate the needle to the shielding position when the safety needle assembly is detached from the medical injector. The needle mount may be positioned so as to rotate around an axis intersecting the longitudinal axis of the tubular housing. The mounting member may be arranged to move axially along the longitudinal axis of the tubular housing. The mounting member may comprise the tubular housing. The mounting member may comprise an inner sleeve section which may be slidably mounted in the tubular housing.

Preferably in the operative position before use, the spring means and the needle mount are arranged at a set position, thereafter use of the safety needle assembly with the medical injector causes a shift from the set position; said shift enables the spring means to rotate the needle to the shielding position when the safety needle assembly is detached from the medical injector.

The needle mount may be arranged to rotate about an axis intersecting the central longitudinal axis of the tubular housing. The needle mount may be arranged to rotate about an axis intersecting a longitudinal axis of the tubular housing which is offset from the central longitudinal axis of the tubular housing. The longitudinal axis of the tubular housing may be central or centrally offset.

Preferably an element is slidably mounted in the tubular housing for movement between an unactivated position and an activated position;
the element including a verification member to audibly verify the safety needle assembly is correctly attached to the medical injector;
the verification member comprising a resilient member which is resiliently flexible between a first configuration and a second configuration;
a deflecting member to flex the verification member during relative movement of the verification member over the deflection member in a distally extending direction;
wherein the resilient member projects axially from the element and attachment of the safety needle assembly to the medical injector causes a part of the medical injector to move the element distally in the tubular housing and said movement causes a part of the resilient member to move over the deflecting member from the first configuration to the second configuration and then back towards the first configuration; and
wherein the verification member is arranged to generate an audible signal as the verification member moves back to the first configuration from the second configuration when the element is moved to the activated position to verify the safety needle assembly is correctly attached to the medical injector.

Preferably the needle mount comprises a unitary component having an integral mounting element or mounting elements in the form of a shaped surface or axial members located on an outer surface. The shaped outer surface may provide spherical surface and may enable the needle mount to be secured by a ball and socket arrangement and may enable a click-fit (ball and socket) arrangement. The axial members may provide hemispherical surfaces and may enable the needle mount to be secured by ball and socket arrangements and may enable a click-fit (ball and socket) arrangement.

Preferably the shift from the set position preloads the spring means to ensure movement of the needle to the shielding position when the safety needle assembly is detached from the medical injector.

The use of the safety needle assembly with the medical injector may comprise the attachment of the safety needle assembly to the medical injector and/or the use thereof and/or the detachment of the safety needle assembly from the medical injector.

The use of the safety needle assembly with the medical injector may cause the spring means and the needle mount to move relative to one another to enable the spring means to rotate the needle to the shielding position when the safety needle assembly is detached from the medical injector.

Preferably the shift from the set position preloads the spring means to ensure movement of the needle to the shielding position (when the safety needle assembly is detached from the medical injector).

Preferably the shift from the set position causes the spring means to generate a force sufficient to urge movement of the needle to the shielding position. Preferably the use of the safety needle assembly with the medical injector causes a change in position of the spring means relative to the needle mount. Preferably the use of the safety needle assembly with the medical injector causes a change in position of the needle mount relative to the spring means. Preferably the use of the safety needle assembly with the medical injector causes the spring means and the needle mount to shift apart. Preferably the use of the safety needle assembly with the medical injector causes the spring means and the needle mount to shift closer together. Preferably the spring means and the needle mount are arranged to shift from the set position to an active position.

Preferably in the set position, the needle mount is retained in the tubular housing with the needle in the operative position. Preferably in the set position the spring means is not fully loaded and may be unloaded. In the set position, the spring means may be partially loaded or completely unloaded.

Preferably in the active position the needle mount is retained in a pivoting position so as to be rotatable around an axis intersecting the longitudinal axis of the tubular housing and the spring means is arranged to urge rotation of the needle about said axis. Preferably in the active position the spring means is fully loaded.

The needle mount may be in the pivoting position with the spring means and needle mount in the set position. The spring means may be arranged to be moved relative to the needle mount from the set position to an active position during attachment of the safety needle assembly to the medical injector. The spring means may be arranged to be moved with the control member.

The needle mount may initially (in the operative position before use) be retained in a non-pivoting position prior to moving to the pivoting position. The non-pivoting position may be defined by a first pair of axial members on the tubular housing. The pivoting position may be defined by a second pair of axial members on the tubular housing. The needle mount may be arranged to move from the first pair of recesses to the second pair of recesses in order to shift the spring means and needle mount from the set position. The needle mount may comprise axial members located on an outer surface. The axial members may each provide hemispherical surfaces. The axial members may be arranged to move from the first pair of recesses to the second pair of recesses.

The axial members of the needle mount may comprise recesses and wherein the needle mount may comprise axial members providing hemispherical surfaces located on an outer surface and wherein the axial members of the needle mount may be arranged to move from the first pair of recesses to the second pair of recesses.

The spring means may be mounted to the tubular housing. The spring means may comprise a leaf spring which may be in the form of a resilient finger. The leaf spring may project inwardly from an inner surface of the tubular housing. The leaf spring may project inwardly perpendicularly relative to the inner surface of the tubular housing. The leaf spring may project inwardly at an acute angle relative to the inner surface of the tubular housing. The leaf spring may project inwardly at an acute angle in a distal or proximal direction relative to the longitudinal internal axis of the tubular housing. The leaf spring may have a curved or arcuate or straight form. The leaf spring may have curved and/or arcuate and/or straight sections.

The movement of the tubular housing relative to the needle mount may shift the spring means and needle mount from the set position and may preload the spring means.

The tubular housing may move proximally (relative to the needle mount) in order to shift the spring means and needle mount from the set position. The tubular housing may move towards the medical injector to shift the spring means and needle mount from the set position. The tubular housing may move distally (relative to the needle mount) in order to shift the spring means and needle mount from the set position.

The spring means may be integral to a mounting member which may comprise an inner sleeve section and/or (a part of) the tubular housing and the combination of the spring means (leaf spring) and the mounting member may comprise a unitary component. The movement of the mounting member relative to the needle mount may shift the spring means and needle mount from the set position. The mounting member may move proximally (relative to the needle mount) in order to shift the spring means and needle mount from the set position. The mounting member may move distally (relative to the needle mount) to shift the spring means and needle mount from the set position.

The blocking means may comprise a control member slidably displaceable along the longitudinal axis of the tubular housing from a set position. The control member may be slidably displaceable along the longitudinal axis of the tubular housing from a set position whereat the control member engages the needle mount to maintain the needle in an operative position. The control member may be arranged to move away from the set position to disengage the needle mount.

The blocking means may comprise a portion of the spring means which engages with the needle mount to prevent rotational/pivotal movement of the needle mount in the set position and, preferably, with the spring in an unloaded position. A tip of the spring means (leaf spring) may locate within an indent defined on an outer surface of the needle mount. The non-pivoting position may be defined by a first pair of axial members on the tubular housing and the indent may locate in the same plane as the first pair of axial members. The depth of the indent and/or length of the resilient finger (spring means) may cause the spring means to be partially loaded in the set position. The depth of the indent and/or the (relative) length of the resilient finger (spring means) may cause the spring means to be completely unloaded in the set position.

The releasable blocking means may be arranged to prevent rotational/pivotal movement of the needle from the operative position whereat the needle extends in a direction along a longitudinal axis of the tubular housing and whereby release of the blocking means allows rotational/pivotal movement of the needle to the shielding position.

The spring means may comprise a leaf spring. The spring means may comprise a plastics material. The spring means may comprise a non-metal material.

An end of the leaf spring may contact an outer surface of the needle mount. Preferably the end of the leaf spring is arranged to move along the outer surface of the needle mount as the spring means and needle mount shift from the set position.

The needle mount may comprise a flange located on an outer surface. The flange may be arranged to contact the spring means in order to transfer the spring force to the needle mount.

The leaf spring may be initially spaced apart from the flange. The leaf spring may move into contact with a flange defined on the needle mount in order to shift the spring means and needle mount from the set position and preferably to an active position and may move the spring to a loaded position. The leaf spring may move into contact with a proximal surface of the flange in order to shift the spring means and needle mount from the set position and preferably to an active position and may move the spring to a loaded position. The leaf spring may move into contact with a distal surface of the flange in order to shift the spring means and needle mount from the set position and preferably to an active position and may move the spring to a loaded position.

Preferably the axis of rotation is statically fixed relative to the tubular housing and the non-patient end moves in a circumferential path about the axis of rotation from the operative position to the shielding position.

The axis of rotation may be radial with respect to the longitudinal axis of the tubular housing. The axis of rotation may be perpendicular with respect to the longitudinal axis of the tubular housing. The axis of rotation may be at an oblique angle with respect to the longitudinal axis of the tubular housing. The axis of rotation may extend across an internal diameter of the tubular housing.

The axis of rotation may extend along a chord across the internal area of the tubular housing.

Preferably the needle mount is co-axial with the tubular housing when the needle is in the operative position.

In the shielding position, the non-patient end of the needle may contact or be adjacent to an internal peripheral wall of the tubular housing. In the shielding position, the spring means may urge the non-patient end of the needle against the internal peripheral wall of the tubular housing.

Preferably, in the operative position, the spring mean has stored energy therein.

In the operative position, the spring means may be latched by a latch mechanism. Preferably the shift from the set position causes the latch mechanism to latch the spring means in a preloaded position to ensure movement of the needle to the shielding position when the safety needle assembly is detached from the medical injector. Preferably the latch mechanism latches the spring means in a loaded position to ensure movement of the needle to the shielding position when the safety needle assembly is detached from the medical injector. The shift from the set position to an active position may be permanent and this may ensure movement of the needle to the shielding position when the safety needle assembly is detached from the medical injector. Preferably the latch mechanism holds and/or maintains the spring means and the needle mount at an active position.

The spring means may be mounted on (or located adjacent to) an internal wall of the tubular at a first side/portion and wherein the non-patient end of the cannula is arranged to locate adjacent to a second side/portion of the internal wall of the tubular housing. The first side/portion may be an opposite side portion provided on the tubular housing relative to the second side portion. The first side/portion may be 180 degrees offset around the tubular housing relative to the second side/portion. The first side/portion may be longitudinally aligned with the second side/portion.

The spring means may contact the needle mount at a position located distally of the axis of rotation. The spring means may contact the needle mount at a position located proximally of the axis of rotation.

Preferably the control member comprises an engagement aperture and a part of the needle mount is arranged, in the set position, to locate within the engagement aperture to prevent rotation of the needle from the operative position. The control member may comprise an annular member and the engagement aperture is located centrally on the annular member. The needle mount may comprise a collar which locates within an aperture of the control member in the set position.

Preferably a distal end of the needle mount provides an outer surface complementary with the engagement aperture of the control member.

The control member may comprise a complementary end face with an end face of a shielding sleeve. Preferably a distal end face of the control member is complementary with a proximal end face of a shielding sleeve.

Preferably the spring means comprises a resilient member which extends inwardly from the tubular housing and contacts an outer surface of the needle mount at a position offset from the axis of rotation and preferably applies a rotational force to rotate the needle mount.

Preferably the spring means comprises a leaf spring projecting inwardly from the tubular housing and, with the needle in an operative position, the leaf spring is in a preloaded condition and may be deflected from a neutral/relaxed position. Preferably with the needle in the operative position the leaf spring has energy stored therein. An end surface of the leaf spring may contact an outer longitudinal surface of the needle mount to create a torque about the axis of rotation.

Preferably the needle mount comprises a unitary component having axial members located on an outer surface. The axial member may provide hemispherical surfaces and may enable the needle mount to be secured by ball and socket arrangements and may enable by a click-fit (ball and socket) arrangement. The tubular housing may comprise axial members located on an internal surface for cooperation with the axial members provided on the needle mount. Preferably the axial members of the needle mount and the tubular housing enable a push fit engagement of the needle mount into the pivoting position within the axial members of the tubular housing. The axial members of the tubular housing and the needle mount may comprise a first pair of axial member comprising projecting portions and a second pair of axial members comprising corresponding recesses. The projecting portions may comprise hemi-spherical projections. The axial members may provide a ball and socket joint. Preferably the axial members provide a first ball and socket joint on one side of the needle mount and a second ball and socket joint on an opposite side of the needle mount for connection to complementary ball and socket joints provided within the tubular housing.

Preferably the control member is mounted to move away from the proximal end (and/or towards the distal end) of the tubular housing in order to disengage the needle mount. The control member may be mounted to move towards the proximal end (and/or away from the distal end) of the tubular housing in order to disengage the needle mount.

Preferably the safety needle assembly comprises a needle shielding sleeve and, in which, the control member may comprise a distal end face of the needle shielding sleeve which provides an or the engagement aperture to prevent rotation of the needle from the operative position whilst the control member is in the set position.

The control member may comprise a disc, the disc being located within a needle shielding sleeve of the safety needle assembly and wherein the disc is slidably displaceable in the needle shielding sleeve from the set position and wherein the control member is frictionally engaged in the needle shielding sleeve and is maintained in a position located adjacent to a distal end of the needle shielding sleeve on movement thereto by the tubular housing during an injection.

The control member may comprise a distal end face and a skirt portion extending therefrom, the distal end face comprising an aperture for engaging an outer surface of the needle mount in the set position and the skirt providing a contact face which is contacted by part of the medical injector and the control member is moved from the set position on attachment of the needle safety assembly to the medical injector.

The control member may be mounted to move towards the proximal end of the tubular housing in order to disengage the needle mount. A needle shielding sleeve may encompass and shield the patient end of the needle in a non-injecting configuration and in which the needle shielding sleeve is co-axial with the tubular housing and may be arranged to slidably move longitudinally relative thereto. A needle shielding sleeve may encompass and shield the patient end of the needle in a non-injecting configuration and, in which, the needle shielding sleeve comprise release means to slidably displace the control member from the set position.

According to a second aspect of the present invention there is provided a drug delivery device comprising a safety needle assembly and a medical injector, the safety needle assembly being in accordance the first aspect of the present invention.

Preferably the medical injector comprises a rubber seal and attachment of the safety needle assembly to the medical injector causes the non-patient end of the needle to pierce and penetrate through the rubber seal and wherein the position of the non-patient end though the rubber seal maintains the needle in an operative position after the control member has moved from the set position.

According to a third aspect of the present invention there is provided a method of shielding a non-patient end of a needle in a safety needle assembly for use with a medical injector, the safety needle assembly being in accordance with the first aspect of the present invention, the method being characterised by;
using the safety needle assembly with the medical injector to shift the mounting member and the needle mount from the set position, said shift moves the mounting member relative to the needle mount to enable the spring means to rotate the needle to the shielding position when the safety needle assembly is detached from the medical injector, and
detaching the safety needle assembly from the medical injector and rotating the needle with the spring means to the shielding position.

The method may comprise moving one of the mounting member or the needle mount relative to the other to enable the spring means to rotate the needle to the shielding position when the safety needle assembly is detached from the medical injector.

According to another aspect of the present invention there is provided a safety needle assembly for use with a medical injector comprising;
a tubular housing removably attachable to the medical injector, the tubular housing extending in a longitudinal direction from a proximal end to a distal end, the proximal end being arranged for attachment to the medical injector;
a needle mount located within the tubular housing for directly or indirectly supporting a double ended needle having a patient end and a non-patient end;
an element slidably mounted in the tubular housing for movement between an unactivated position and an activated position;
the element including a verification member to audibly verify the safety needle assembly is correctly attached to the medical injector;
the verification member comprising a resilient member which is resiliently flexible between a first configuration and a second configuration;
a deflecting member to flex the verification member during relative movement of the verification member over the deflection member in a distally extending direction;
wherein the resilient member projects axially from the element and attachment of the safety needle assembly to the medical injector causes a part of the medical injector to move the element distally in the tubular housing and said movement causes a part of the resilient member to move over the deflecting member from the first configuration to the second configuration and then back towards the first configuration; and
wherein the verification member is arranged to generate an audible signal as the verification member moves back to the first configuration from the second configuration when the element is moved to the activated position to verify the safety needle assembly is correctly attached to the medical injector.

Preferably the audible signal is generated at a position coincident to the element arriving at the activated position.

Preferably the verification member is arranged to generate an audible signal as the verification member moves back to the first configuration from the second configuration when the element arrives at the activated position to verify the safety needle assembly is correctly attached to the medical injector.

The audible signal may comprise a single audible signal. The single audible signal may comprise a single click.

The audible signal may comprise a series of audible signals. The series of audible signals may comprise a series of clicks.

The resilient member may project axially from the element in a proximal direction. The resilient member may project axially from the element in a distal direction

Preferably the audible signal provides a dual function in providing an audible signal to verify that the safety needle assembly is correctly attached to the medical injector (a non-patient end of the cannula is correctly positioned in the medical injector) and also verifies that the spring means is preloaded. Preferably the audible signal provides a dual function in providing an audible signal to verify that the safety needle assembly is correctly attached to the medical injector and also verifies the shift from the set position to an active position which may be permanent.

The element may comprise a disc member. The element may comprise an annular member. The element may comprise a ring member. The element may comprise a sleeve section or tubular section.

The element may be arranged to move axially along the longitudinal axial axis of the tubular housing. Preferably the element is arranged to move distally along the longitudinal axis of the tubular housing. Preferably the element is arranged to slidably move without any rotational movement.

Preferably the element comprises a proximal surface which is arranged for contact with a (distal) part of the medical injector such that (distal) movement of the medical injector relative to the tubular housing causes corresponding movement of the element relative to the tubular housing.

Preferably the verification member comprises a resilient member which may comprise a resilient finger or a resilient skirt. Preferably the resilient member extend axially in a proximal or distal direction. The verification member may project/extend (axially) forwardly/distally from a front surface of the element. The verification member may project/extend (axially) rearwardly/proximally from a rear surface of the element. The verification member may comprise a head located at an end thereof. The head may provide a shaped surface to cooperate with a surface of the deflecting member. The cooperating surface may permit one way movement of the verification member relative to the deflecting member thereby forming a latch arrangement.

Preferably the deflecting member is defined on an inner surface of the tubular housing. The deflecting member may comprise a lug or rib which projects inwardly from the inner surface of the tubular member. The deflecting member may have a distal surface which may comprise a ramped surface. The deflecting member may comprise a proximal surface which may comprise an abutment surface. The abutment surface may provide an abutment surface extending directly radially inwardly in a generally perpendicular direction.

The audible signal may comprise a click.

Preferably the verification member latches the element in a latched position relative to the tubular housing. Preferably in the latched position a spring urges the needle to move from an operative position to a shielding position. Preferably attachment of the safety needle assembly to the medical injector causes the spring to move from an unloaded position to a loaded position. Preferably the position that the spring moves from the unloaded position to the loaded position is coincident with the position that the element is latched and preferably this is also coincident with the generation of the audible signal. Accordingly, the audible signal may alert the user to both the correct attachment of the safety needle assembly to the medical injector and the latching of the element in the tubular housing (and the spring is loaded).

After the injection has been performed, the latched position of the element maintains the loaded position of the spring which continues to urge the needle to the shielding position. After detachment of the medical injector from the safety needle assembly, the latched position of the element maintains the loaded position of the spring which continues to urge the needle to the shielding position.

### BRIEF DESCRIPTION OF THE DRAWINGS

The present invention will now be described, by way of example only, with reference to the drawings that follow, in which:

FIG. 1 is an exploded view of an embodiment of a safety needle assembly together with a medical injector.

FIG. 2 is a perspective view of a tubular housing, a needle mount and a needle of an embodiment of a safety needle assembly.

FIG. 3 is a partial cut away view of a first preferred embodiment of a safety needle assembly with the spring and needle mount in the set position.

FIG. 4 is a partial cut away view of a first preferred embodiment of a safety needle assembly detached from a medical injector in a used configuration.

FIG. 5 is a partial cut away view of a second preferred embodiment of a safety needle assembly with the spring and needle mount in the set position.

FIG. 6 is a partial cut away view of a third preferred embodiment of a safety needle assembly with the spring and needle mount in the set position.

FIG. 7 is a partial cut away view of a third preferred embodiment of a safety needle assembly attached to a medical injector ready to perform an injection.

FIG. 8 is a partial cut away view of a third preferred embodiment of a safety needle assembly attached to a medical injector after an injection.

FIG. 9 is a partial cut away view of a third preferred embodiment of a safety needle assembly detached from a medical injector in a used configuration.

FIG. 10 is a partial cut away view of a sixth preferred embodiment of a safety needle assembly with the spring and needle mount in the set position and including a first embodiment of a verification mechanism prior to activation.

FIG. 11 is a partial cut away view of a seventh preferred embodiment of a safety needle assembly with the spring and needle mount in the set position and including a second embodiment of a verification mechanism prior to activation.

FIG. 12 is a partial cut away view of a seventh preferred embodiment of a safety needle assembly being attached to a medical injector and including a second embodiment of a verification mechanism prior to activation.

FIG. 13 is a partial cut away view of an eighth preferred embodiment of a safety needle assembly detached from a medical injector and including a third embodiment of a verification mechanism after activation.

FIG. 14 is a partial cut away view of a ninth preferred embodiment of a safety needle assembly prior to the coupling/uniting of a needle mount in the tubular housing.

FIG. 15 is a partial cut away view of a ninth preferred embodiment of a safety needle assembly with the needle mount coupled/united in the tubular housing.

FIG. 16 is a cross section of part of a tenth preferred embodiment of a safety needle assembly with the spring and needle mount in the set position.

FIG. 17 is a cross section of part of a tenth embodiment of a safety needle assembly detached from a medical injector in a used configuration.

FIG. 18 is a cross section of an eleventh embodiment of a safety needle assembly.

### DESCRIPTION OF THE PREFERRED EMBODIMENTS

As shown in the figures and as will be described, the proximal end of the safety needle assembly attached to a distal end of a medical injector 11. The distal end of the safety needle assembly 10 will be pressed against the skin of patient during an injection and the distal end of the medical injector 11 locates away for the patient.

In this specification, the references to using and the use of the safety needle assembly covers the attachment of the safety needle assembly to the medical injector and/or injecting a patient and delivering the liquid medicament and/or the detachment of the safety needle assembly from the medical injector.

The pen needle 12 is a double ended needle 12 and includes a patient end 14 or distal end comprising a sharp tip for insertion into a patient. The double ended needle 12 also includes a non-patient end 15 or proximal end comprising a sharp tip for insertion into a drug vial or cartridge 16 provided by the pen injector 11. The proximal/non-patient end 15 of the needle will typically have to pierce a rubber seal 18 which may comprise a septum or stopper provided on the end of the vial or cartridge 16 to access the liquid medicament or drug contained within the cartridge.

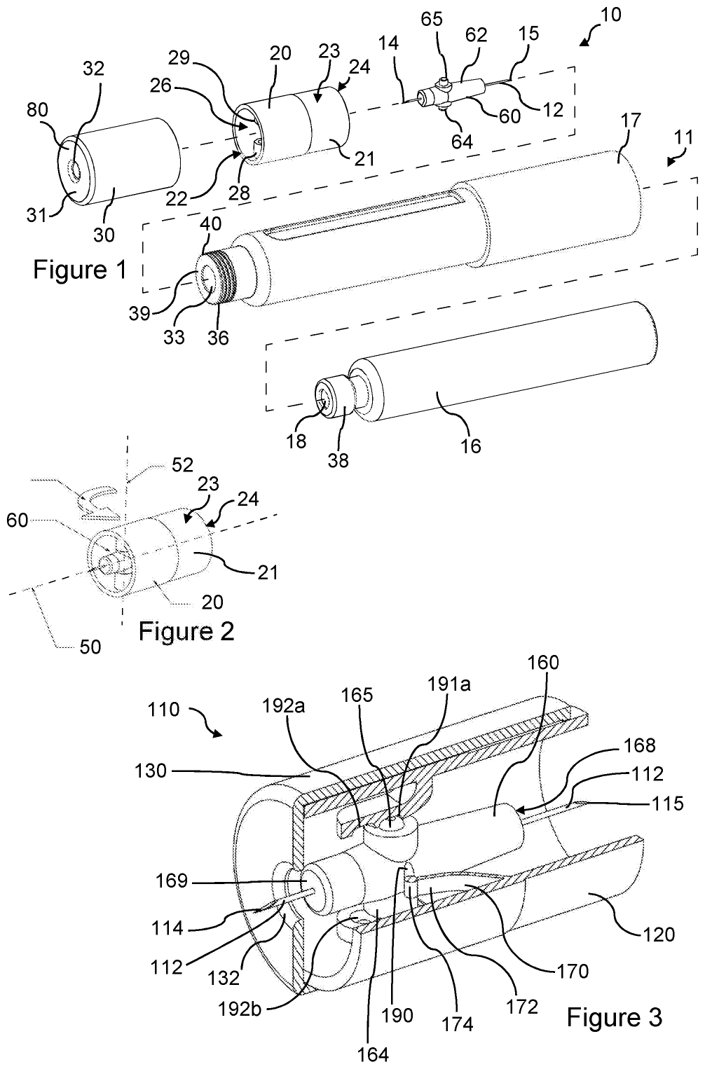

With reference to FIG. 1, a safety pen needle assembly 10 and a medical injector 11 are shown in an exploded view. The safety needle assembly 10 generally includes a hub or tubular housing 20, a needle 12, a shielding sleeve 30. The tubular housing 20 includes a body 21 extending in a longitudinal direction from a proximal end 24 to a distal end 22. The proximal end 24 is formed with an open face open and is shaped to receive a portion of the injector 11 to allow the attachment of the safety needle assembly 10 to the injector 11. For the description of the present invention, the term cannula 12 will now be used to specifically refer to the double ended needle of the safety needle assembly 10. The cannula 12 includes a distal end 14 formed for insertion into a patient, and a proximal end 15 for communication with a liquid medicament located in a reservoir. As shown in the figures, the distal end 14 of the cannula 12 protrudes distally beyond the distal end 22 of the tubular housing 20. The proximal end 15 of the cannula 12 may be within the interior of the tubular housing 20 adjacent to the proximal end 24, or may protrude/extend proximally from the proximal end 24. The cannula 12 is mounted within a needle mount 60 and may be fixed therein using any known technique, such as being adherently fixed to the needle mount 60.

As shown in FIG. 1, a drug delivery device in the form of a medical pen or injector pen comprises a safety needle assembly 10 which is arranged, in use, to be attached to the medical injector 11. The medical injector 11 comprises a cartridge 16 containing a medicament/fluid and, in particular, the cartridge 16 contains a volume of liquid medicament.

The present invention provides a safety needle assembly 10 which is attachable to the medical injector 11. The safety needle assembly 10 provides the cannula 12 through which the liquid medicament is delivered from the cartridge 16 to the patient. The cannula 12 comprises a double ended needle having a sharp tip at the patient end (distal end) 14 and also a sharp tip at the non-patient end (proximal end) 15. However, it should be appreciated that the terms needle and cannula are used interchangeably and the present invention may also be suitable for other types of drug delivery devices 8.

In order to deliver the liquid medicament, the cannula 12 is held in an operative position. In this operative position, the linear cannula 12 protrudes directly out of the end of the drug delivery device and, specifically, the cannula 12 extends along the (central) longitudinal axis 50 of the tubular housing 20. Accordingly, in this operative position, the cannula 12 is located in a central co-axial position within the tubular housing 20. This co-axial position enables the cannula 12 to protrude perpendicularly from a front face 31 of the needle safety assembly 10.

The safety assembly 10 provides a front face 31 having a central window or aperture 32 through which the cannula 12 projects or is projectable. The safety needle assembly 10 is provided with an end cap in the form of a shielding sleeve 30 which includes the central window 32. The shielding sleeve 30 or end cap functions as a blocking means in the form of a control member 80 to retain the position of the cannula 12 and to release the cannula 12.

The control member 80 or shielding sleeve 30 is mounted to the tubular housing 20. The control member 80 or shielding sleeve 30 locates over the outer surface 23 of the tubular housing 20 and is retained thereto. However, control member 80 or shielding sleeve 30 is slidably movable in a longitudinal direction relative to the tubular housing 20.

The housing 17 of the injector 11 has a distal end providing an external screw thread 36 to engage with an internal screw thread provided on the safety needle assembly 10. Specifically, the proximal end 24 of the tubular housing 20 provides the internal screw thread for attaching the safety needle assembly 10 to the housing 17.

The cannula 12 is secured within a needle mount 60. The needle mount 60 is arranged to be fixed within the tubular housing 20 in order to secure the cannula 12 within the safety needle assembly 10. The needle mount 60 is provided with two axial members in the form of lugs 64, 65 for engagement within two corresponding axial members in the form of recesses 28, 29 provided internally on the tubular housing 20. In particular, the tubular housing 20 defines a rotational axis which may be a fixed rotational axis about which the cannula 12 is able to rotate (or pivot). Such movement is functionally important and enables the cannula 12 to rotate from an operative position to a shielding position. In the shielding position, the non-patient end 15 of the cannula 12 is shielded to prevent and/or inhibit back end needle stick injuries. A fixed rotational axis may be provided by axle elements which are engaged within corresponding recesses to allow the rotational/pivoting action of the needle mount 60 relative to the tubular housing 20. In alternative embodiments, the rotational/pivoting functionality may be provided by a flexible section such as a live hinge arrangement whereby the rotational axis may not be statically fixed to solely provide rotation about a static rotational axis.

The cannula 12 is arranged to rotate (or pivot) about a fixed axis 52 (or fixed point) in the tubular housing 20. In the operative position, the cannula 12 extends along the central longitudinal axis of the tubular housing 20. The fixed axis 52 for rotation extends perpendicularly and intersects the central longitudinal axis 50. The fixed axis 52 extends diametrically across the tubular housing 20 and extends radially from the central longitudinal axis 50. This creates a fixed axis 52 or fixed pivoting point or fixed point of rotation for the cannula 12 which is located on the central longitudinal axis 50.

The cannula 12 is arranged to rotate about the fixed axis 52 from an operative position in which the cannula 12 extends along the central longitudinal axis 50 to a shielding position in which the cannula 12 extends at an angle to the central longitudinal axis 50. In particular, in the shielding position, the cannula 12 extends in a direction oblique to the central longitudinal axis 50.

Due to the fixed axis 52 and fixed point of rotation for the cannula 12, the movement from the central longitudinal axis 50 to an oblique position causes the non-patient end 15 of the cannula 12 to move towards the internal wall 26 of the tubular member 20. In this position with the non-patient end 15 of the cannula 12 towards and adjacent to the internal wall 26 of the tubular housing 20, the non-patient end 15 is shielded to a greater degree and provides significantly reduced risk of creating a needle stick injury. Furthermore, as a result of the fixed axis 52, the pivoting action of the needle about the fixed point located on the central longitudinal axis 50 means that the non-patient end 15 of the cannula 12 also moves forwardly (in a distal direction) and away from the open end 24 of the tubular housing 20. This movement of the non-patient end 15 of the cannula 12 in a distal direction of the longitudinal axis 50 of the tubular housing 20 thereby increases the distance between the open end 24 of the tubular housing 20 and the non-patient end 15 of the cannula 12. This increases the level of protection afforded by increasing the distance by which a tip of a finger would need to be inserted into the open end 24 of the tubular housing 20 in order to make contact with the non-patient end 15 of the cannula 12. This increased separation distance coupled with the non-patient end 15 also being located adjacent to the wall 26 of the tubular housing 20 provides increased safety for users from needle stick injuries.

The cannula 12 is secured within the needle mount 60 which provides two axle lugs 64, 65 on opposing sides. The axle lugs 64, 65 are received within two corresponding recesses 28, 29 in the tubular housing 20 such that the cannula 12 is rotatable about a fixed axis 52 within the tubular housing 20.

The safety needle assembly 10 is attached to the pen injector 11 using the screw threads 36 provided on the end of the pen injector 11 and also on the tubular housing 20. As the safety needle assembly 10 is brought into engagement with the pen injector 11 the non-patient end 15 of the cannula 12 abuts, pierces and then projects through the rubber seal 18. During this attachment, the cannula 12 is held along the central longitudinal axis 50 in order to assist with the smooth movement of the cannula 12 through the rubber seal 18.

The tubular housing 20 includes spring means in the form of a leaf spring in the form of a resilient finger which projects inwardly from the internal surface 26 of the tubular housing 20. The resilient finger includes a contact tip which contacts or abuts an outer surface of the needle mount body 62 at a position located offset from the fixed axis 52 of rotation defined by the axial members 28, 29, 64, 65. Accordingly, in the operative position, the resilient finger is in a configuration whereby the tip exerts a force, specifically a moment or torque, on the outer surface of the needle mount 60. Accordingly, in the operative position, the resilient finger is not in a neutral position and is in a deflected state relative to the neutral relaxed condition/state/position. In particular, in the operative position, the leaf spring/resilient finger is preloaded such that energy is stored within the leaf spring/resilient finger. This stored energy acts to urge the cannula 12 away from the operative position and away from being aligned with the longitudinal axis of the tubular housing 20. Before assembly of the needle mount 60 within the tubular housing 20, the leaf spring/resilient finger is in a neutral state with no stored energy. On mounting of the needle mount 60 within the tubular housing 20, the leaf spring/resilient finger is deflected and moved to store energy which acts to urge the needle mount 60 away from the operative position. On release of the cannula 12/needle mount 60 by both the control member 80 and the rubber seal 18, the non-patient end 15 of the cannula 12 locates adjacent to the internal wall 26 of the tubular housing 20. In this configuration, the leaf spring/resilient finger would typically not be the neutral position and energy is still stored such that the leaf spring/resilient finger continues to press and urge the non-patient end 15 of the cannula 12 against the internal wall 26 of the tubular housing 20.

The needle mount 60 is maintained in the operative position by control means which engages a part of the needle mount 60 and maintains the cannula 12 in the operative position. In the first preferred embodiment, the blocking means is provided by the shielding sleeve 30 which includes the central aperture 32. The central aperture 32 engages around a front end of the needle mount 60. In addition, the shielding sleeve 30 is secured around the tubular housing 20 and is only able to move longitudinally with respect to the tubular housing 20 in a distal direction. Accordingly, in the set position, the front end of the needle mount 60 is secured within the aperture 32 of the shielding sleeve 30 such that any force exerted by the resilient finger is counteracted and does not cause rotation of the needle mount 60 or cannula 12 about the fixed axis 52.

In this set position, it can be seen that the patient end 14 of the needle protrudes/projects outwardly from the drug delivery device and is thereby able to inject a patient and to deliver the liquid medicament from the pen injector 11. Once the injection has been completed, the shielding sleeve 30 is moved forwardly to a shielding position. The specific details of the arrangement of the front shielding sleeve 30 are not provided in this specification and there are many suitable devices available, for example as described in WO2011/092518. In this position, the needle mount 60 is no longer engaged within the aperture 32 of the shielding sleeve 30 and can no longer prevent the spring means rotating the needle mount 60 about the fixed axis 52. However, the non-patient end 15 of the cannula 12 remains engaged within the rubber seal 18 along the longitudinal axis 50 such that this arrangement still prevents rotation of the needle mount 60 about the fixed axis 52.

After use the safety needle assembly 10 requires disposal and is, therefore, detached from the medical injector 11 through use of the threaded surfaces 36 to detach and unscrew the safety needle assembly 10. On detachment, the non-patient end 15 of the cannula 12 is withdrawn back through and disengaged from the rubber seal 18 and is therefore no longer held by the rubber seal 18 along the longitudinal axis 50 of the tubular member 20. In this configuration, there is no restraining means preventing rotation of the needle mount 60 due to the exerted torque of the resilient finger and, therefore, the needle mount 60 automatically rotates about the fixed axis 52.

The energy stored in the resilient finger rotates the needle mount 60 about the fixed axis 52 until the non-patient end 15 of the cannula 12 locates adjacent to and, preferably, abuts the internal side wall 26 of the tubular housing 20. The length of the cannula 12 ensures that the non-patient end 15 abuts the internal side wall 26. As a result of the fixed axis 52 of the needle mount 60, the non-patient end 15 of the cannula 12 moves in a distal direction and circumferentially about the axis 52 in an arcuate direction such that the distance along the longitudinal extent between the non-patient end 15 and the open face 24 of the tubular housing 20 increases. Accordingly, not only does the non-patient end 15 locate adjacent to the internal wall 26 of the tubular housing 20 but the non-patient end 15 is also withdrawn/retracted into the tubular housing 20 which significantly increases the protection afforded from needle stick injuries. Both the sideways, radial movement of the non-patient end 15 and the retraction/withdrawal movement in the distal direction of the non-patient end 15 are performed automatically and simultaneously to provide improved passive needle stick protection which requires no intervention by the user. Furthermore, in the shielding position, the leaf spring may continue to exert a pivoting/rotating force on the needle mount 60 and this presses the non-patient end 15 of the cannula 12 against the internal wall 26 of the tubular housing 20.

The operation and use of further embodiments of suitable safety needle assemblies are shown and described in GB2104342.7 which is incorporated herein by reference.

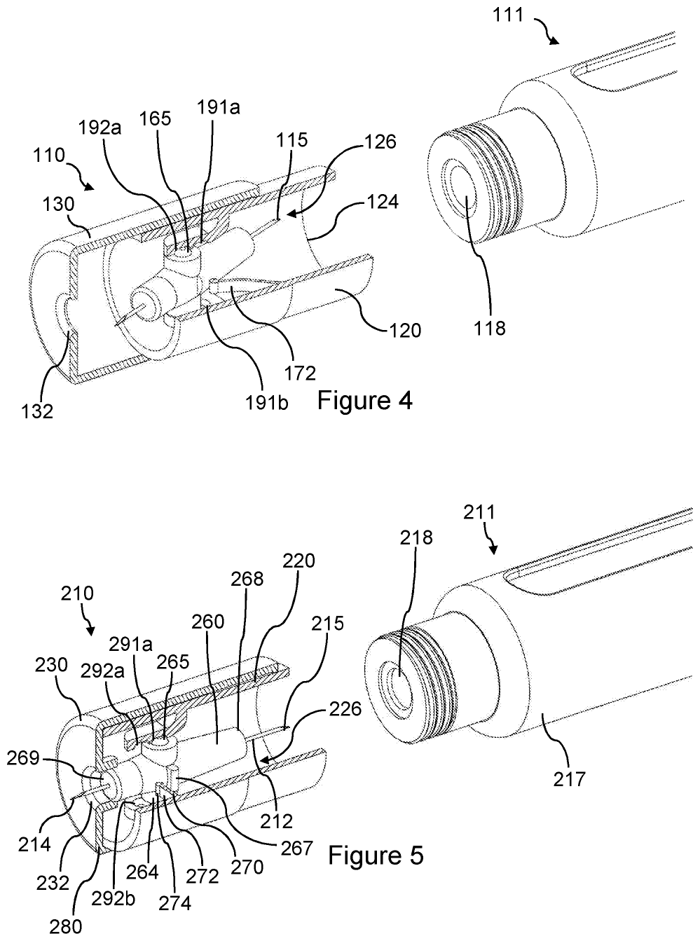

A first embodiment of the present invention is shown in FIG. 3 and FIG. 4. The safety needle assembly comprises a needle/cannula 112 mounted in a tubular housing 120 which provides a mounting member for the spring 170. The cannula 112 is located within the needle mount 160 which is located within the tubular housing 120. In this embodiment, the shielding sleeve 130 provides the blocking means which retains the alignment of the cannula 110 along the longitudinal axis of the tubular member 120. In particular, the blocking means is arranged to counteract the pivoting force of the spring or an ability of the cannula to freely rotate in order to retain the cannula 112 in an operative position. In this embodiment, the spring 170 (leaf spring) is integral with the tubular housing 120 (mounting member) which moves relative to the needle mount 160. The spring 170 and the tubular housing 170 form a unitary component.

The spring 170 and the needle mount 160 are arranged at a set position relative to each other and the use of the safety needle assembly 110 with the medical injector 111 causes a shift from the set position and the shift moves one of the mounting member or the needle mount relative to the other to enable the spring means to rotate the needle to the shielding position when the safety needle assembly is detached from the medical injector. In some embodiments, both the needle mount and the mounting may actually/physically move although this is covered by the use of the term relative movement. This shift enables the spring 170 to then rotate the needle 112 to the shielding position when the safety needle assembly 110 is detached from the medical injector 111. In the first, second and third embodiments, the shift is caused by the attachment of the safety needle assembly 110 to the medical injector 111. In the fourth and fifth embodiments, the shift is caused by the use of the device i.e. the act of injecting the patient. As it will be appreciated, the shift could also be caused by the detachment (in particular, the initial part of the detachment sequence) of the safety needle assembly 110 from the medical injector 111.

FIG. 3 shows the safety needle assembly 110 in an initial position with the spring 170 and the needle mount 160 in the set position prior to attachment to the medical injector 111. The tubular housing 120 provides axial members for engaging axial members of the needle mount 160. The axial members 191a, 191b, 192a, 192b of the tubular housing 120 comprise recesses 191a, 191b, 192a, 192b and the axial members of the needle mount comprise axle lugs 164, 165. In particular, the tubular housing 120 provides a first pair of recesses 191a, 191b to mount the needle mount 160 at a first position (FIG. 3) and a second pair of recesses 192a, 192b to mount the needle mount 160 at a second position.

The needle mount 160 is arranged to be moved relative to the spring 170 from the first (set) position to the second (active) position during the attachment of the safety needle assembly 110 to the medical injector 111. In the set position, the spring 170 may be in an unloaded position by which the spring 170 is not able to pivot the needle mount 160. For example, the spring 170 may be completely unloaded with no stored force being exerted on the needle mount 160, or the spring 170 may only be partially loaded and/or in combination with a contact position on the needle mount 160 which results in the spring 170 not being able to pivot the needle mount. In the active position, the load in the spring 170 and/or the contact position on the needle mount 160 are arranged such that the spring 170 is able to pivot the needle 112 although such movement may not actually occur until the blocking means is disengaged and/or the non-patient end 115 of the cannula 112 is extracted from the rubber seal 118.

As previously described, the safety needle assembly 110 is attached to the medical injector 111 through cooperating threaded surfaces 136, 137 or a similar arrangement. During this attachment, the non-patient end 115 of the cannula 112 pierces the rubber seal 118. This penetration eventually causes a proximal end 168 of the needle mount 160 to contact the rubber seal 118 or the area surrounding the rubber seal 118. This stops and prevents further movement of the needle mount 160 proximally relatively towards the medical injector 111.

Further engagement of the threaded surfaces 136, 137 continues the movement of the tubular housing 120 towards the medical injector 111. This movement thereby causes the tubular housing 120 to move relative to the needle mount 160 which is prevented from moving further by the end of the medical injector 111. In particular, the axle lugs 164, 165 are caused to move out of the first pair of recesses 191a, 191b and into the second pair of recesses 192a, 192b. In addition, the distal end 169 of the needle mount 160 projects into the aperture 132 of the shielding sleeve 130 to provide the blocking means.

The safety needle assembly 110 comprises spring means 170 in the form of a resilient finger 172 including a tip 174 which contacts and abuts an outer surface of the needle mount 160. In the first embodiment, the resilient finger 172 is provided on an inner surface 126 of the tubular housing 120 and projects inwardly therefrom. Accordingly, since the needle mount 160 moves relative to the tubular housing 120 during attachment, the needle mount 160 also moves relative to the resilient finger 172 during the attachment of the safety needle assembly 110 to the medical injector 111. Specifically, the point of contact between the needle mount 160 and the resilient finger 170 moves along (proximally) the outer surface of the needle mount 160 from a first contact position (FIG. 3) to a second contact position.

As shown in FIG. 3, the needle mount 160 provides a first contact position in the form of a shaped surface comprising a recess or an indent 190. In the first position, the tip 174 of the resilient finger 172 locates within this indent 196. The depth of this indent 190 may result in no or only a minimal transfer of any spring force from the resilient finger 170 to the needle mount 160. In addition, the indent 190 is located diametrically along the support axis provided by the first pair of recesses 191a, 191b. This thereby prevents the resilient finger 172 from urging the needle mount 160 to rotate relative to the tubular housing 120 when the axle lugs 164, 165 are located within the first pair of recesses 191a, 191b, as provided in the first (set) position prior to attachment. Accordingly, this prevents fatigue of the resilient finger prior to use.

In addition, the shape of the indent 190 is arranged to retain the needle mount 160 aligned with the longitudinal axis of the tubular housing 120. As shown in FIG. 3, it can be seen that the needle mount 160 is prevented from rotating/pivoting by the tip 174 of the resilient finger 172. Specifically, the tip 174 will create a counter-rotational force if the needle mount 160 attempts to rotate within the tubular housing 120.

As described above, the needle mount 160 is arranged to move relative to the tubular housing 120 as the safety needle assembly 110 is attached to the medical injector 111. Specifically, a force is created through the contact of the proximal end 168 of the needle mount 160 with the distal end of the medical injector 111. This urges the needle mount forwardly relative to the tubular housing 120 and dislodges the tip 174 of the resilient finger from the indent 190. The tip 174 will then track proximally along the outer surface of the needle mount 160 until the movement is stopped and the axle lugs 164, 165 are retained in the second pair of recesses 192a, 192b in a second (active) position.

At this position, the resilient finger 172 creates a force on the needle mount 160. The point of contact is now offset from the diametrical axis created by the second pair of recesses 192a, 192b and this results in the resilient finger 172 creating a rotational or pivoting force on the needle mount 160. However, the distal end of the needle mount 160 now locates within the aperture 132 of the shielding sleeve 130 which thereby counteracts this rotational force and retains the cannula 112 in an operative position.

The subsequent operation of the present invention is essentially the same as that described above with reference to FIG. 1 and FIG. 2 and as further described in GB2104342.7. Accordingly, the present invention provides an improved safety needle assembly 110 due to the removal of any fatigue on the spring means 170 prior to attachment of the safety needle assembly 110 to the medical injector 111. Furthermore, the activation of the spring means is automatic on attachment of the safety needle assembly 110 to the medical injector 111.

Briefly, the subsequent operation and use of the safety needle assembly 110 after attachment comprises injecting a patient following which the shielding sleeve 130 moves or is moved to an outer distal position. At this position, the distal end 169 of the needle mount 160 is no longer retained within the aperture 132 (blocking means) of the shielding sleeve 130. Accordingly, the engagement/penetration of the non-patient end 115 of the cannula 112 in the rubber seal 118 is the sole means for preventing the spring action of the resilient finger 172 from rotating the needle mount 160 about the axis between the second pair of recesses 192a, 192b.

The safety needle assembly 110 is then detached from the medical injector 111 through the use of the screw threads 136, 137 to the detached position shown in FIG. 4. Accordingly, as the non-patient end 115 of the cannula 112 disengages and is removed from the rubber seal 118, the spring force of the resilient finger 172 automatically and immediately rotates the cannula 112 to a shielding position. In this position, the non-patient end 115 is moved to the inner surface 126 of the tubular housing 120 and is also retracted further into the tubular housing 120 away from the proximal open end 124.

A second embodiment of an improved safety needle assembly 210 is shown in FIG. 5. In this embodiment, the tip 274 of the resilient finger 272 initially locates distally relative to the axis through the axle lugs 264, 265 of the needle mount 260. In addition, the resilient finger 272 extends directly and radially inwardly towards the central longitudinal axis of the tubular housing 220. In the first embodiment, the resilient finger 272 does not extend directly radially inwardly but extends with an internal acute angle relative to the tubular housing 220. In the second embodiment, the resilient finger 272 forms an internal right angle with the tubular housing 220. Again, in this embodiment, the spring 270 (leaf spring) is integral with the tubular housing 220 (mounting member) which moves relative to the needle mount 260. The spring 270 and the tubular housing 270 form a unitary component.

In this embodiment, the distal end 269 of the needle mount 260 is engaged by the shielding sleeve 230 prior to attachment of the safety needle assembly 210 to the medical injector 211. This maintains the cannula 212 aligned with the longitudinal axis of the tubular housing 220. This operative position of the cannula prevents any damage to the cannula and also maintains the non-patient end 215 in the optimum position for subsequently piercing the rubber seal 218.

The safety needle assembly 210 is attached to the medical injector 211 using the screw threads 236, 237 and this causes the proximal end 268 of the needle mount 260 to contact the distal end of the medical injector 211. Subsequent use of the threads 236, 237 causes the tubular housing 220 to keep moving proximally and the needle mount 260 moves relative thereto. This relative movement causes the axle stubs 264, 265 to dislodge/disengage from the first pair of recesses 291a, 291b. The outer ends of the axle stubs 264, 265 are shaped to cause/aid this disengagement and the subsequent re-engagement in the second pair of recesses 292a, 292b. In particular, the outer ends of the stub axles 264, 265 are substantially hemi-spherical. The stub axles 264, 265 re-engage in the second pair of recesses 292a, 292b to provide an operative position for the device. Accordingly, the attachment of the safety needle assembly 210 to the medical injector 211 causes the spring 270 and needle mount 260 to shift from the set position.

During the relative movement between the needle mount 260 to the tubular housing 220, the spring means 270 moves proximally with the tubular housing 220 relative to the needle mount 260. The needle mount 260 provides a flange 267 for engaging with the resilient finger 272. In particular, the tip 274 of the resilient finger 272 contacts and abuts a distal surface of the flange 267. This contact exerts a force on the flange 267 since the resilient finger 272 is in a deformed and loaded position when the axle stubs 264, 265 are engaged in the second pair of recesses 292a, 292b. This loading provides a rotational force for the needle mount 260 since the force is offset relative to the axis of rotation created by the pair of recesses 292a, 292b. However, the distal end 269 of the needle mount 260 is engaged in the shielding sleeve 230 and, also, the non-patient end 215 of the cannula 212 is engaged in the rubber seal 218. Accordingly, the needle mount 260 is not able to rotate but the spring means 270 is loaded. In this embodiment and in some of the other described embodiments, the spring 270 is shown to be completely unloaded in the set position. However, it should be appreciated that the spring 270 may be partially, semi-preloaded in the set position. In the set position, it is advantageous to avoid fully preloading the spring but zero or a small or a partial preloading of the spring 270 still provides the invention with the advantage of extending and prolonging the characteristics of the material of the spring 270.

The patient end 214 of the cannula 212 is ready to perform the injection. Following the injection, the shielding sleeve moves or is moved to a distal position. In this position, the distal end 269 of the needle mount 260 is no longer engaged with the shielding sleeve 230. However, the non-patient end 215 of the cannula 212 remains engaged in the rubber seal 218 such that the cannula 212 is still held in a position aligned with the longitudinal axis of the tubular housing 220.

The detachment of the safety needle assembly 210 from the medical injector 211 causes the non-patient end 215 to be released from the rubber seal. The loading of the resilient finger 272 then causes the needle mount 260 to rotate about the axis created between the second pair of recesses 292a, 292b. The non-patient end 215 of the cannula 212 is withdrawn into the tubular housing 220 and also moves to the inner surface 226 of the tubular housing 220 to prevent needle stick injuries.

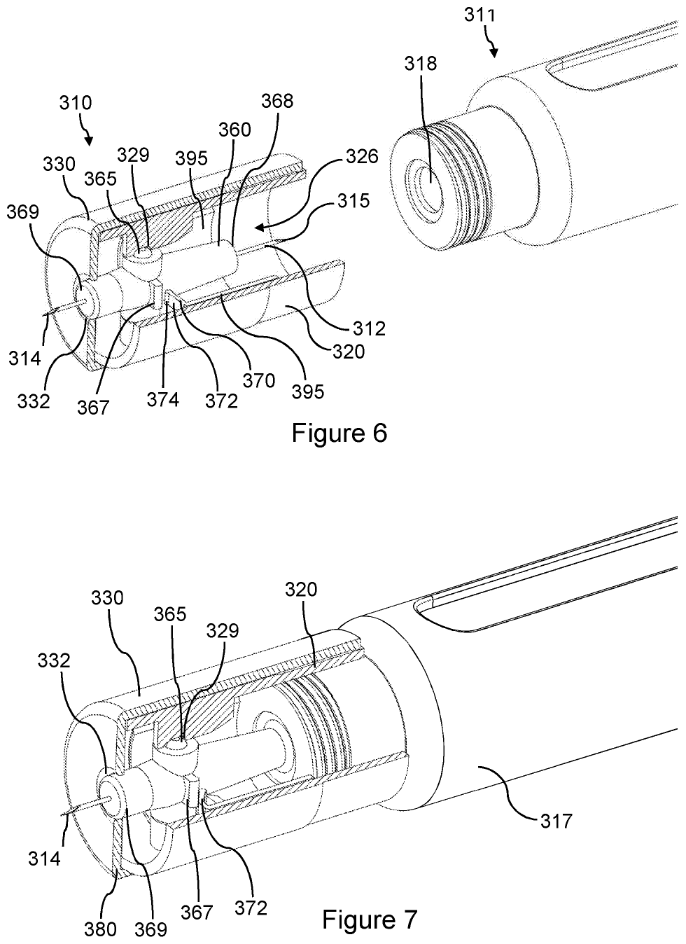

A third embodiment of a safety needle assembly 310 is shown in FIG. 6 to FIG. 9. In this embodiment, the spring means 370 is again provided by an internally projecting resilient finger 372 with a tip 374. However, the resilient finger 372 is located proximally relative to the recesses 328, 329 and the axis of rotation. In the initial position, the spring means 370 is not loaded and the needle mount 360 is held in position due to the distal end 369 of the needle mount 360 being engaged in the aperture 332 of the shielding sleeve 330. In this embodiment, the spring 370 (leaf spring) is integral with an inner sleeve section 395 (mounting member) which moves relative to the needle mount 360. The spring 370 and the tubular housing 370 form a unitary component. The inner sleeve section 395 is axially slidably located within the tubular housing 320.

In this embodiment, the resilient finger 372 is mounted on an inner sleeve section 395 which is a separate and distinct component to the tubular housing 320. The inner sleeve section 395 locates and slides internally relative to the tubular housing 320. The inner sleeve section 395 may have an incomplete cross section and may be substantially C-shaped or may have cut out section(s) to allow for the internally projecting mount(s) for the recesses 328, 329.

In this embodiment, the resilient finger 372 moves distally from an unloaded position (set position) to a loaded position (active position). In FIG. 6, the resilient finger 372 is shown projecting inwardly perpendicularly relative to the inner surface of the tubular housing 320. However, it should be readily appreciated that the resilient finger 372 (leaf spring) may project inwardly perpendicular to or at an acute angle in a distal or proximal direction relative to the longitudinal internal axis of the tubular housing 320. Specifically, the resilient finger 372 (leaf spring) may be projecting inwardly “pointing generally” to the front (distal) or rear (proximal) ends of the housing. Furthermore, the resilient finger 372 may have a curved or arcuate or straight form, or may have curved and/or arcuate and/or straight sections (to perform best as a spring). The needle mount 360 is provided with a flange 367 and the tip 374 of the resilient finger 372 is arranged to contact and create a force on a proximal surface of the flange 367. This force is offset from the axis of rotation of the needle mount 360 provided by the recesses 328, 329. This thereby urges movement of the needle 312 to the shielding position.

In use, the safety needle assembly 310 is provided detached with the spring means 370 in an unloaded position, as shown in FIG. 6. In some embodiments, in the set position, the spring means is partially or semi-preloaded or completely unloaded. Whilst in this detached configuration, it will be readily appreciated that the inner sleeve section 395 is held in position relative to tubular housing 320 in order to maintain the inner sleeve section 395 within the tubular housing 320. Any suitable arrangement may be used to maintain this positional arrangement, for example frictional resistance between the inner sleeve section 395 and the tubular housing 320 (as shown in FIG. 6), a latch mechanism, reciprocal projections/recesses on the inner sleeve section 395 and the tubular housing 320 etc. The safety needle assembly 310 is attached to the medical injector 311, as shown in FIG. 7. This attachment causes the spring means 370 to move distally relative to the needle mount 360 in order to load the spring means 370. Specifically, the attachment of the safety needle assembly 310 to the medical injector 311 causes the spring 370 and needle mount 360 to shift from the set position (to an active position). The mounting member and the needle mount are arranged at a set position relative to each other. Thereafter use of the safety needle assembly with the medical injector causes a shift from the set position; and the shift moves one of the mounting member or the needle mount relative to the other to enable the spring means to rotate the needle to the shielding position when the safety needle assembly is detached from the medical injector. In particular, a proximal end 397 of the inner sleeve section 395 abuts and makes contact with a distal end of the medical injector 311 when partially attached. The inner sleeve section 395 is then prevented from moving any further towards the medical injector 311 but the further attachment moves the tubular housing 320 towards the medical injector 311. Accordingly, the tip 374 of the resilient finger 372 moves towards and abuts the flange 367 and the spring means 370 is loaded producing a rotational force on the needle mount 360. In particular, the spring means is preloaded to generate a force sufficient to enable the spring means to rotate the needle to the shielding position when the safety needle assembly is detached from the medical injector.

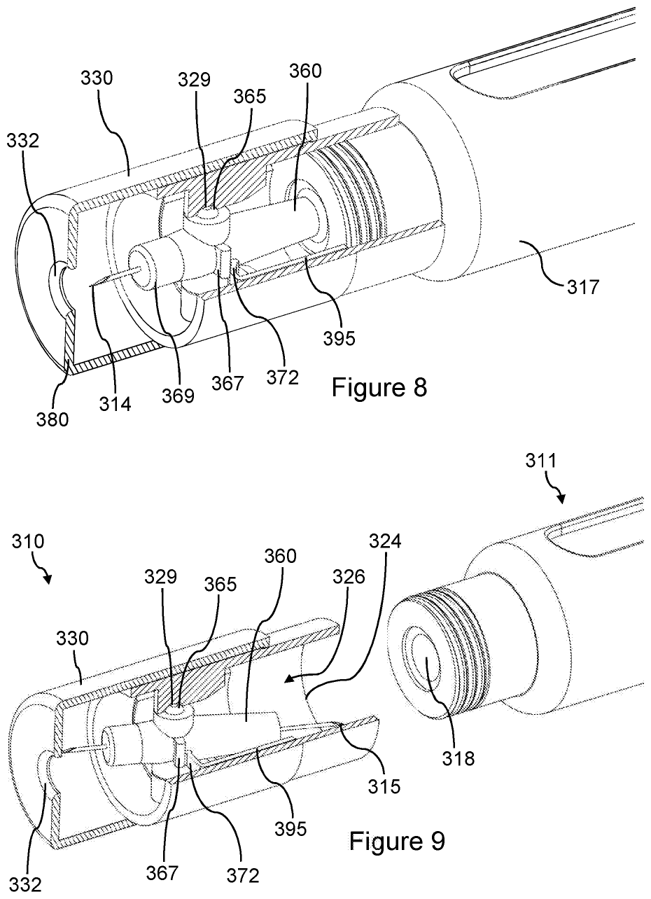

Following the attachment and loading of the spring means 370 (e.g. from a partially or semi-preloaded or completely unloaded state to a (fully) preloaded state), the injection is performed and the shielding sleeve 330 moves or is moved distally to a shielding position, as shown in FIG. 8. In this position, the resilient finger 372 is urging the needle mount 360 to rotate but this is counteracted by the engagement of the non-patient end 315 with the rubber seal 318.

The subsequent detachment of the safety needle assembly 310 from the medical injector 311 releases this counteraction engagement. In particular, the non-patient end 315 is released from the rubber seal 318 and the needle mount 360 immediately rotates due to the loading of the resilient finger 372. The non-patient end 315 is withdrawn into the tubular housing 320 and locates/abuts the internal surface 326 of the tubular housing in a safe condition. It will be readily appreciated that the inner sleeve section 395 is held in position relative to the needle mount 360 in order to maintain the urging force exerted by the resilient finger on the flange 367. Any suitable arrangement may be used to maintain this force, for example frictional resistance between the inner sleeve section 395 and the tubular housing 320 (as shown in FIG. 9), a latch mechanism, reciprocal projections/recesses on the inner sleeve section 395 and the tubular housing 320 etc. By way of confirmation, the shift from the set position (to an active position) is permanent to ensure movement of the needle 312 to the shielding position when the safety needle assembly 310 is detached from the medical injector 311. Therefore, frictional contact (as shown in the drawings) or some other means would be required to hold and maintain the inner sleeve section 395 within the tubular housing 320 at an active position.

In a fourth embodiment of a safety needle assembly, the resilient finger locates distally relative to the flange on the needle mount and move proximally from the unloaded position to the loaded position. The loading of the spring means is caused by the movement of the shielding sleeve and the attachment of the safety needle assembly to the medical injector does not shift the spring and needle mount from the set position. In this embodiment, the spring is integral with an inner sleeve section and forms a unitary component. As the shielding sleeve moves proximally, the inner distal end surface of the shielding sleeve contacts a distal end of the inner sleeve section and the inner sleeve section and the resilient finger move proximally relative to the needle mount which is held statically within the recesses of the tubular housing. This relative movement causes the tip of the resilient finger to contact the flange provided on the needle mount in order for the spring means to be loaded. Specifically, the use and the act of injecting the patient shifts the spring and needle mount from the set position. The shielding sleeve then moves to an outer distal position which releases the distal end of the needle mount. In this position the engagement of the cannula at the non-patient end solely counteracts the rotational force being exerted on the needle mount by the spring means. Finally, the safety needle assembly is detached from the medical injector and positions the non-patient end against the inner surface of the tubular housing.

In a fifth embodiment, again it is the subsequent movement of the shielding sleeve that shifts the spring and needle mount from the set position and activates the loading of the spring means. Furthermore, in this embodiment, the loading is due to the provision of two pairs of recesses. The stub axles are engaged within a first pair of recesses and a distal collar on the needle mount. In this embodiment, the spring is integral with the tubular housing and forms a unitary component. The movement of the shielding sleeve by the skin of the patient thereby causes the shielding sleeve to contact and moves the distal collar and hence the needle mount from the first (distal) pair of recesses to a proximal pair of recesses and hence rearwardly towards the medical injector. In this position, the spring means is loaded but the needle mount is restrained. As before, the shielding sleeve moves or is moved to an outer shielding position and releases the needle mount but the cannula is still engaged with the rubber seal to prevent rotation of the needle mount. Finally, the safety needle assembly is detached from the medical injector which releases the cannula from the rubber seal and the loaded resilient finger causes the needle mount to immediately rotate.

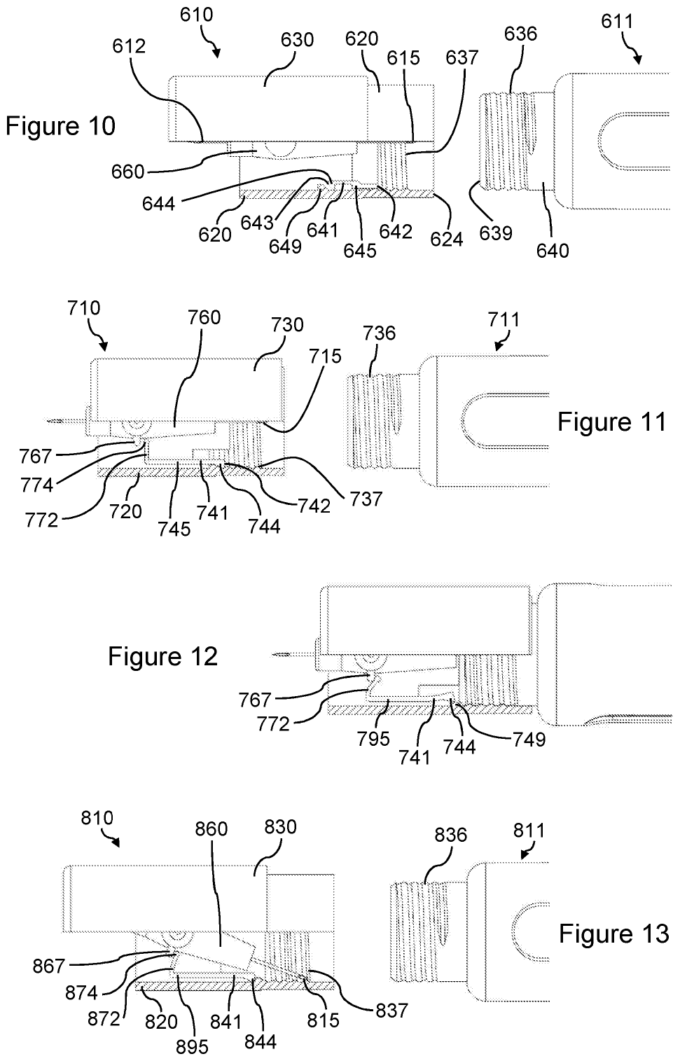

A sixth embodiment of the present invention is shown in FIG. 10. This embodiment provides a verification mechanism or verification system which verifies that the safety needle assembly 610 is correctly positioned on the medical injector 611. In particular, the verification mechanism provides feedback to the user who is manually attaching the safety needle assembly 610 to the medical injector 611. The safety needle assembly 610 may be the same as that described in relation to embodiments one to five. However, the verification system may also be used with other safety needle assemblies and medical injectors.

In the sixth embodiment, the verification mechanism provides an audible signal or audible indicia to alert the user that the correct attachment position of the safety needle assembly 610 on the medical injector 611 has been reached. This audible alert may comprise a click or another suitable audible signal. In particular, the safety needle assembly 610 is screwed on the medical injector 611 using the corresponding threads. This moves the safety needle assembly 610 towards an attached position and, in the correct attached position, the non-patient end 615 of the cannula 612 is safely located and positioned within the chamber of the medical injector 611. Accordingly, this verifies to the user that the device is ready for use and prevents any risk of the safety needle assembly 610 only being position in an attached position but with the non-patient end of the cannula 612 not being in the desired position.

The verification mechanism comprises an element 645 which is (axially) movably located within the tubular housing 620. In particular, the element 645 is arranged to slidably move within the tubular housing 620 between an unactivated position and an activated position at which point an audible signal is generated. The element 645 includes a proximal end 642 which is arranged for contract with the distal face 639 of the medical injector 611 during attachment of the safety needle assembly 610 to the medical injector 611.

As shown in FIG. 10, the medical injector 611 has a boss 640 which has an external screw thread 636 (threaded surface) provided around the periphery. The tubular housing 620 provides a corresponding internal screw thread 637 (threaded surface) in order for the medical injector 611 to be attached to the safety needle assembly 610.

The medical injector 611 is screwed relatively on to the safety needle assembly 610 and this action causes the distal end face 639 to initially contact and abut the proximal end 642 of the element 645. The further screwing action thereby causes the element 645 to move with the distal end 639 such that this causes the element 645 to slidably move within the tubular housing 620 towards a distal end of the tubular housing 620.

In this embodiment, the element 645 provides a verification member which is a resilient member. The resilient member extends axially from the element 645. This enables a greater force to be generated in a resilient finger 641 to create a reliable and/or louder audible noise compared to a radially extending tab which may not generate an audible signal at all due to the amount of deflection that would be available. Such a small distance of deflection would not reliably generate enough force to create an audible signal to be relied upon. The resilient member may extend either in a proximal direction or a distal direction from the element 645. In this embodiment, the resilient member is in the form of a distally/forwardly extending latch finger 641 which has a distal latching head 644. The latch finger 641 has a proximal end 643 which secures the latch finger 641 to the element 645. The latch finger 641 is integral with the element and forms a unitary component. The latch finger 641 comprises a resilient material which enables the latch finger 641 to be flexed inwardly.

The tubular housing 620 provides a deflecting member in the form of a lug 649 or rib or other suitable latching surface on an inner surface. The lug 649 includes a ramped/angled proximal surface which is arranged to deflect the latch finger 641 inwardly. The lug 649 comprises a distal surface which is arranged to engage with the latch head 644 so as not to deflect the latch finger 641 inwardly and this prevents disengagement of the latch finger 641 from the lug 649 once engaged.

As the element 645 is moved distally due to the relative screwing action, the latch head 644 reaches the ramped surface and this causes the latch finger 641 to flex inwardly. Further screwing action then causes the latch finger 641 to move over a top edge of the lug 649 and the latch finger 641 naturally flexes back outwardly to a latched position. This movement of the latch finger 641 causes an audible signal to be emitted due to the contact of the latch finger 641 with the tubular housing 620. This audible signal is sufficient to alert a user to the engagement of the latch finger 641. Due to the relative position of the lug 649 within the safety needle assembly 610, the position at which the latch finger 641 engages is coincident with the correct relative position of the safety needle assembly 610 and the medical injector 611. In this correct attached position, the non-patient end of the cannula 615 is optimally located within the medical injector 611.

The audible signal both provides confirmation the safety needle assembly 610 is correctly attached to the medical injector 611 and prevents the user from over tightening/positioning and under tightening/positioning the safety needle assembly 610 and the medical injector 611. As it will be appreciated, an under connection may result in the non-patient end 615 of the cannula 612 not locating correctly in the medical injector 611. Similarly, an over connection may create unnecessary stresses and cause failures/fractures in the material of the safety needle assembly 610 or the medical injector 611. Accordingly, this audible alert, which may comprise a click or a similar noise, reliably informs the user of a correctly connected safety needle assembly 610 which is then ready for use as previously described.

In some embodiments, the latching movement may also provide a tactile sensation providing feedback to the user to reaffirm and verify/confirm the audible signal heard by the user. For example, a vibration will resonate through the safety needle assembly 610 which is being held by the fingertips of a user. This feedback may help to confirm the audible signal to the user.

In some embodiments, the element 645 may comprise or be integral with or be connected to an inner sleeve section 395, 495 as described in previous embodiments and may include the integral leaf spring mounted thereon. In particular, with the element shown in FIG. 10, the element may form a part of the inner sleeve section 395 shown in FIG. 6 to FIG. 9. In this arrangement, the verification mechanism would provide a dual function in providing the audible signal to demonstrate the correct attachment position and also would form the retaining mechanism to maintain/hold the inner sleeve section 395 within the tubular housing 320 at an active position (shown in FIG. 9).

A seventh embodiment of the present invention is shown in FIG. 11 and FIG. 12. The embodiment again provides a verification mechanism or verification system which verifies that the safety needle assembly 710 is correctly positioned on the medical injector 711. In particular, the verification mechanism provides feedback in the form of an audible signal (for example, a click) to the user who is manually attaching the safety needle assembly 710 to the medical injector 711. The safety needle assembly 710 may be the same as that described in relation to embodiments one to five. In particular, the seventh embodiment is effectively similar to the third embodiment shown in FIG. 6 to FIG. 9.

In this embodiment, the verification mechanism comprises latching means which is provided at a proximal end 742 of a proximally extending latching finger 741. The latching finger extends axially and, specifically, proximally from the element 745 conversely compared to the sixth embodiment. In particular, the latching finger 741 extends rearwardly (proximally) rather than forwardly (distally). The tubular housing 720 provides a lug 749 or other suitable latching surface on an inner surface. The lug 749 includes a ramped/angled proximal surface which is arranged to deflect the latch finger 741 inwardly. The lug 749 comprises a distal surface which is arranged to engage with the latch head 744 so as not to deflect the latch finger 741 inwardly and this prevents disengagement of the latch finger 741 from the lug 749 once engaged.

The element 745 is initially positioned in the tubular housing 720 with the head 744 located adjacent to the proximal ramped surface of the lug 749 such that the head 744 does not move over the threads 737 during attachment of the safety needle assembly 710 to the medical injector 711. The lug 749 is positioned such that the latching of the head 744 is coincident with the correct engagement of the safety needle assembly 710 and the correct loading of the needle mount 760 with the spring means (resilient finger 772). The movement of the head 744 over the lug 749 to the latched position creates/generates an audible signal (for example a click) to demonstrate the attachment of the medical injector 711 to the safety needle assembly 710. A tactile signal which can be sensed (by the touch of the user) may also be generated simultaneously and naturally with the audible signal.

The verification mechanism also forms the retaining mechanism to maintain/hold the inner sleeve section 795 within the tubular housing 720 at an active position (see the equivalent earlier embodiment shown in FIG. 9). In particular, the corresponding profiles of the head 744 of the latching finger 741 and/or the lug 749 causes the latching means to solely hold the inner sleeve section 795 in the active position with the head or tip 774 of the resilient finger 772 pressed against the flange 767 to keep the non-patient end of the cannula 715 withdrawn into the tubular housing 720 and locating/abutting the internal surface 726 of the tubular housing 720 in a safe condition. In this embodiment, the spring 772 is integral with the inner sleeve section 795 and forms a unitary component.

The verification mechanism comprises the inner sleeve section 795 which provides an element 745 movably located within the tubular housing 720. In particular, the element 745/inner sleeve section 795 is arranged to slidably move within the tubular housing 720. The element 745/inner sleeve section 795 includes a proximal end 797 (provide by the head 744) which is arranged for contract with the distal face 739 of the medical injector 711 during attachment of the safety needle assembly 710 to the medical injector 711.

The medical injector 711 is screwed relatively on to the safety needle assembly 710 and this action causes the distal end face 739 to initially contact and abut the proximal end 797 of the element 745/inner sleeve section 795. The further screwing action thereby causes the element 745/inner sleeve section 795 to move with the distal end 739 such that this causes the element 745/inner sleeve section 795 to slidably move within the tubular housing 720 towards a distal end of the tubular housing 720.

As mentioned above, in this embodiment, the element 745/inner sleeve section 795 has a proximally extending latching finger 741 with the latching head 744 defined at the proximal end. The latching head 744 comprises an outwardly shaped portion with an outer peripheral surface to cooperate with the lug 749. The latching finger 741 is integral with the element 745/inner sleeve section 795 and forms a unitary component. The latching finger 741 comprises a resilient material which enables the latching finger 741 to be flexed inwardly. The lug 749 provides a suitable latching surface on an inner (distal) surface to prevent movement of the latching finger 741 proximally, once engaged/latched.

As the element 745/inner sleeve section 795 is moved distally due to the relative screwing action, the distal surface of the head 744 reaches the ramped surface of the lug 749 and this causes the latching finger 741 to flex inwardly. Further screwing action then causes the latching finger 741 to move over the lug 749 with the latching finger 741 naturally flexing back outwardly after moving over the lug 749. This movement of the latching finger 741 causes an audible signal to be emitted due to the sudden contact of the latching finger 741 with the lug 749/outer tubular member 720. This audible signal is sufficient to alert a user to the engagement process of the latching finger 741. Due to the relative position of the lug 749 within the safety needle assembly 710, the position at which the latching finger 741 engages with the lug 749 is coincident with a correct relative position of the safety needle assembly 710 and the medical injector 711. In this correct attached position, the non-patient end of the cannula 715 is optimally located within the medical injector 711. Since the latching movement emits an audible signal or click, the user will know when the correct attachment has occurred. In this position, the proximal end 724 of the tubular housing 720 locates adjacent to a shoulder 748 of the medical injector 711 or, in some embodiments, the tubular housing 720 may abut the shoulder 748 of the medical injector 711. The verification mechanism provides a dual function in providing an audible signal to demonstrate the correct attachment position (non-patient end of the cannula 715 positioned in the medical injector 711) and also demonstrates that the spring means (resilient finger 772) is preloaded against the flange 767.

An eighth embodiment (FIG. 13) again provides a verification mechanism which verifies that the safety needle assembly 810 is correctly positioned on the medical injector 811. The verification mechanism comprises latching means which is secured around and/or integral with the inner sleeve section 895. The inner sleeve section 895 has a latching skirt 841 extending therefrom which deflects/flexes inwardly over the thread 837 as the medical injector 811 is attached to the safety needle assembly 810. The latching skirt 841 provides the proximal end for contacting the medical injector 811 during attachment. As the latching skirt 841 moves over the threads 837, an audible signal (for example a click) or a series of audible signals (for example clicks) is/are created to demonstrate the attachment of the medical injector 811 to the safety needle assembly 810. The latching skirt 841 extends axially from the element/inner sleeve section 895 and, in particular, extends proximally from the inner sleeve section 895. The spring 872 is integral with the inner sleeve section 895 and forms a unitary component.

In this embodiment, the inner sleeve section 895 has a proximal skirt 841 with a latching rim 844 defined around the proximal edge. The latching rim 844 comprises an outwardly shaped portion with an outer peripheral surface to cooperate with the threads 837. The latching skirt 841 is integral with the inner sleeve section 895 and forms a unitary component. The latching skirt 841 comprises a resilient material which enables the latching skirt 841 to be flexed inwardly.

The threads 837 and, in particular, the final thread of the tubular housing 820 provide a suitable latching surface on an inner surface to prevent or at least inhibit movement of the latching skirt 841 proximally. The threads 837 provide a generally sinusoidal surface which is arranged to deflect the latching skirt 841 inwardly during movement of the inner sleeve in a distal direction. However, the latching rim 844 provides a proximal surface which is arranged to engage with threads 837 so as not to deflect the latching skirt 841 inwardly and this prevents disengagement of the latching skirt 841 from the final thread 837 once engaged.

As the inner sleeve section 895 is moved distally due to the relative screwing action, the distal surface of the latching rim 844 reaches the ramped surface of the first thread 837 and this causes the latching skirt 841 to flex inwardly. Further screwing action then causes the latching skirt 841 to move the subsequent threads 837 with the latching skirt 841 naturally flexing back outwardly after moving over each peak of the threads 837. This movement of the latching skirt 841 causes an audible signal to be emitted due to the sudden contact of the latching skirt 841 with the troughs of the threads 837. This audible signal is sufficient to alert a user to the engagement process of the latching skirt 841. Due to the relative positions of the threads 837 within the safety needle assembly 810, the position at which the latching skirt 841 engages in the threads 837 is coincident with a correct relative range of positions of the safety needle assembly 810 and the medical injector 811. In this correct attached position, the non-patient end of the cannula 815 is optimally located within the medical injector 811. Since the threads 837 will emit a number of audible signals or clicks, the user may know to screw the medial injector 811 until a number of clicks have been heard and the further screwing action is prevented due to the end of the threads. In this position, the proximal end 824 of the tubular housing 820 locates adjacent to a shoulder 848 of the medical injector 811 or, in some embodiments, the tubular housing 820 may abut the shoulder 848 of the medical injector 811.

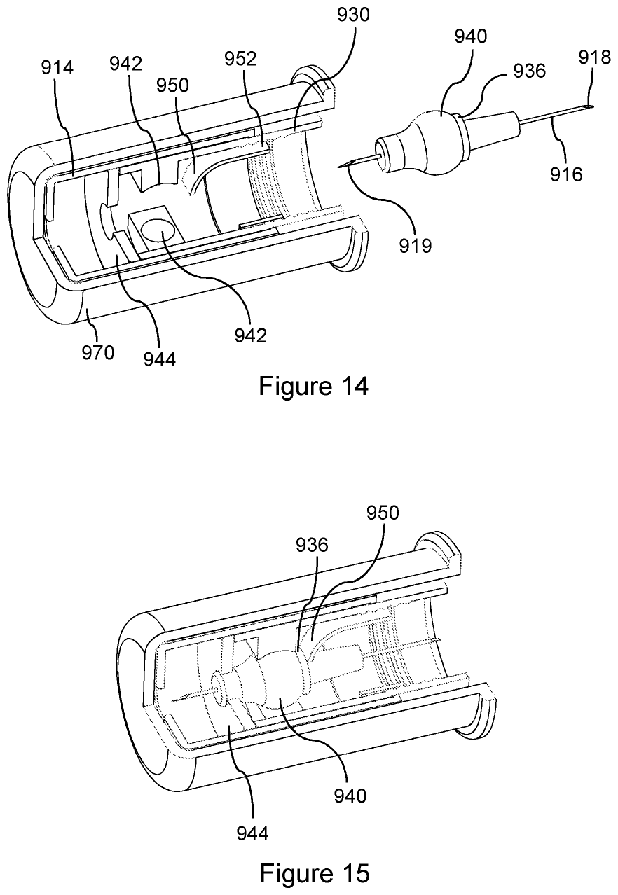

A ninth preferred embodiment of a safety needle assembly 910 is shown in FIG. 14 and FIG. 15. The needle mount 940 is positioned within the tubular housing 930 in a fixed position and the spring 950 is located on an inner sleeve section 952 which is movable within the tubular housing 930. Prior to insertion of the needle mount 140 (see FIG. 14), it will be readily appreciated that the inner sleeve section 952 is held in position relative to tubular housing 930 in order to maintain the inner sleeve section 952 within the tubular housing 930. Any suitable arrangement may be used to maintain this positional arrangement, for example frictional resistance between the inner sleeve section 952 and the tubular housing 130, a latch mechanism, reciprocal projections/recesses, detents etc. on the inner sleeve section 952 and the tubular housing 930 etc. The tubular housing provides two axially opposed recesses 942 (mounting members) which retain the needle mount 940 in a fixed longitudinal position but enables the needle mount 940 to articulate within the mounting members 942. This mounting arrangement provides a ball and socket type joint. The needle is initially held along the central longitudinal axis by a blocking member 944. The spring 950 is integral with the inner sleeve section 952.

The attachment of the safety needle assembly 910 to the medical injector causes the spring 950 (mounted on the inner sleeve section 952) to move distally relative to the needle mount 940 in order to load the spring 950. The tip of the spring 950 moves towards and abuts the flange 936 and the spring 950 is loaded generating a rotational force on the needle mount 940. The injection is performed and the shield 914 (outer shield) moves or is moved distally back to a shielding position. During the injection procedure, the blocking means 944 (control member) moves distally and remains at the distal end of the shield 914. In this position, the blocking means 944 no longer maintains the needle 916 along the central longitudinal axis. In this position, the spring 950 is urging the needle mount 940 to rotate but this is counteracted by the engagement of the non-patient end 918 with the rubber seal. The subsequent detachment of the safety needle device 910 from the medical injector releases this counteraction engagement and the needle mount 940 immediately rotates due to the loading of the spring 950. It will be readily appreciated that the inner sleeve section 952 is held in position relative to the needle mount 940 in order to maintain the urging force exerted by the spring on the flange 936. Any suitable arrangement may be used to maintain the inner sleeve section 952 in position, for example frictional resistance between the inner sleeve section 952 and the tubular housing 930, a latch mechanism, reciprocal complementary projections/recesses/detents or cam profiles on the inner sleeve section 952 and the tubular housing 930 etc. By way of confirmation, the shift from the set position (to an active position) is permanent to ensure movement of the needle 916 to the shielding position when the safety needle device 910 is detached from the medical injector 912.

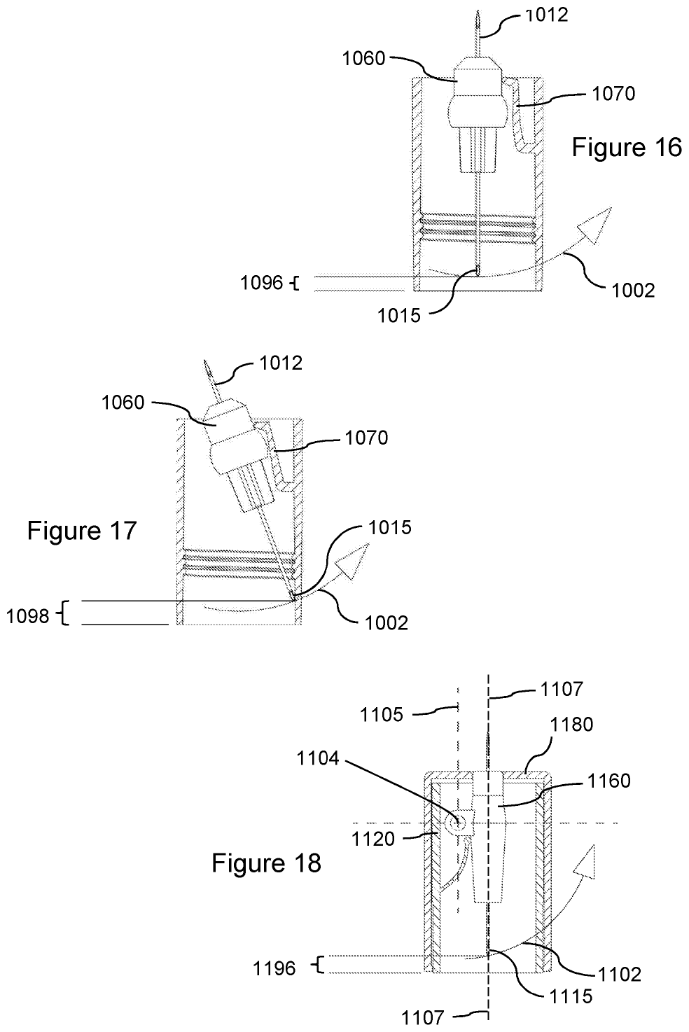

In a tenth embodiment of the present invention, the needle mount 1060 comprises a spherical outer surface and the integral leaf spring 1070 acts/contacts a forward/distal portion of the needle mount 1060, as shown in FIG. 16 and FIG. 17. In this embodiment, the non-patient end 1015 of the cannula 1012 is again moved to a shielding position. In the shielding position, the non-patient end 1015 of the cannula 1012 locates along the same side of the tubular housing as the leaf spring 1070. It will be appreciated that if the leaf spring 1070 contacts the needle mount 1060 at a position distal/forward of the axis then the leaf spring 1070 (integral with the tubular housing) will move the needle mount 1060 in an anti-clockwise direction 1002 with reference to FIG. 16 and FIG. 17. Conversely, if the leaf spring 1070 did contact the needle mount 1060 at a position proximal/rearwardly relative to the axis of rotation then the needle mount 1060 is urged in a clockwise direction. In this embodiment, the non-patient end 1015 of the cannula 1012 travels around the circumferential/arcuate path 1002 and the separation distance increases from a first distance 1096 to a second distance 1098.

Finally, an eleventh embodiment of the present invention is shown in FIG. 18 which shows that the needle mount 1160 is arranged to rotate about an axis 1104 which is located offset from the central longitudinal axis 1107 of the tubular housing 1120 which has an integral spring. An outer surface 1161 of the front portion of the needle mount 1160 may again be arranged to be engaged by the control member 1180. The axis of rotation 1104 still locates along a longitudinal axis 1105 of the tubular member but this longitudinal axis not is not the central longitudinal axis of the tubular housing 1120. In this embodiment, it can be seen that the offset location of the axis or rotation 1104 can increase the retraction distance 1196, 1198 for the non-patient end 1115 of the cannula 1112 on moving to the shielding position. This results from the increase in the length of the arcuate travel path 1102 of the non-patient end 1115 and this thereby increases the final separation distance 1198.

1. A safety needle assembly for use with a medical injector comprising;
a tubular housing removably attachable to the medical injector, the tubular housing extending in a longitudinal direction from a proximal end to a distal end, the proximal end being arranged for attachment to the medical injector;
a needle mount located within the tubular housing for directly or indirectly supporting a double ended needle having a patient end and a non-patient end, the needle mount being arranged to allow movement of the needle from an operative position to a shielding position;
a spring means to urge movement of the needle to the shielding position;
a releasable blocking means arranged to prevent movement of the needle from the operative position whereat the needle extends in a direction along a longitudinal axis of the tubular housing and whereby release of the blocking means allows movement of the needle to the shielding position;
the needle mount is a unitary component inserted into the tubular housing and positioned so as to rotate around an axis and the spring means is arranged to urge rotation of the needle about said axis; and
the blocking means engages the needle mount to maintain the needle in the operative position before use, thereafter attachment of the safety needle assembly to the medical injector and the use thereof causes the blocking means to disengage from the needle mount and allow the spring means to rotate the needle to the shielding position and to move the non-patient end of the needle towards the distal end of the tubular housing whereat the needle extends at an angle oblique to the longitudinal axis of the tubular housing to place the non-patient end of the needle at a location adjacent to an interior wall of the tubular housing;
characterised in that;
the safety needle assembly further comprises:
a mounting member which moves relative to the needle mount and wherein the spring means is integral to the mounting member, and
in the operative position before use, the mounting member and the needle mount are arranged at a set position relative to each other, thereafter use of the safety needle assembly with the medical injector causes a shift from the set position; said shift moves one of the mounting member or the needle mount relative to the other to preload the spring means to generate a force sufficient to enable the spring means to rotate the needle to the shielding position when the safety needle assembly is detached from the medical injector.

2. A safety needle assembly according to claim 1 in which the mounting member is arranged to move proximally relative to the needle mount in order to cause the shift from the set position to and active position to preload the spring means to generate a force sufficient to enable the spring means to rotate the needle to the shielding position when the safety needle assembly is detached from the medical injector.

3. A safety needle assembly according to claim 1 in which the mounting member is arranged to move distally relative to the needle mount in order to cause the shift from the set position to an active position to preload the spring means to generate a force sufficient to enable the spring means to rotate the needle to the shielding position when the safety needle assembly is detached from the medical injector.

4. A safety needle assembly according to claim 1 in which the needle mount comprises a unitary component having an integral mounting element in the form of a shaped surface on an outer surface and wherein the shaped outer surface provides spherical surface.

5. A safety needle assembly according to claim 4 in which the spherical surface enables the needle mount to be secured by a ball and socket arrangement within the tubular housing.

6. A safety needle assembly according to claim 4 which the spherical surface enables a click-fit (ball and socket) arrangement.

7. A safety needle assembly according to claim 1 in which the needle mount comprises a unitary component having integral mounting elements in the form of shaped axial members located on an outer surface and wherein the axial members provide hemispherical surfaces and enable the needle mount to be secured by ball and socket arrangements.

8. A safety needle assembly according to claim 1 in which the use of the safety needle assembly with the medical injector causes a change in position of the spring means relative to the needle mount.

9. A safety needle assembly according to claim 1 in which, in the set position, the spring means is partially or semi-preloaded or completely unloaded.

10. A safety needle assembly according to claim 1 in which the needle mount is positioned so as to rotate around an axis intersecting the longitudinal axis of the tubular housing.

11. A safety needle assembly according to claim 1 in which the mounting member is arranged to move axially along the longitudinal axis of the tubular housing.

12. A safety needle assembly according to claim 1 in which the spring means comprises a leaf spring and an end of the leaf spring contacts an outer surface of the needle mount and wherein the end of the leaf spring is arranged to move along the outer surface of the needle mount as the spring means and needle mount shift from the set position.

13. A safety needle assembly according to claim 1 in which the needle mount comprises a flange located on an outer surface and wherein the flange is arranged to contact the spring means in order to transfer the spring force to the needle mount.

14. A drug delivery device comprising a safety needle assembly and a medical injector, the safety needle assembly being in accordance with claim 1.

15. A method of shielding a non-patient end of a needle in a safety needle assembly for use with a medical injector, the safety needle assembly being in accordance with claim 1, the method being characterised by;
using the safety needle assembly with the medical injector to shift the mounting member and the needle mount from the set position to an active position, said shift moves the mounting member relative to the needle mount to enable the spring means to rotate the needle to the shielding position when the safety needle assembly is detached from the medical injector, and
detaching the safety needle assembly from the medical injector and rotating the needle with the spring means to the shielding position.

16. A safety needle assembly according to claim 1 in which the mounting member comprises an inner sleeve section which is slidably mounted in the tubular housing.

17. A safety needle assembly according to claim 1 in which the spring means is integral to the mounting member which comprises an inner sleeve section and the combination of the spring means and the mounting member comprises a unitary component.

18. A safety needle assembly according to claim 1 in which the spring means is integral to the mounting member which comprises a part of the tubular housing and the combination of the spring means and the mounting member comprises a unitary component.

19. A safety needle assembly according to claim 1 in which the movement of the mounting member relative to the needle mount shifts the spring means and needle mount from the set position.

20. A safety needle assembly according to claim 19 in which the mounting member moves distally relative to the needle mount to shift the spring means and needle mount from the set position.
